(19) United States
(12) Patent Application Publication
(10) Pub. No.: US 2026/0248501 A1
(43) Pub. Date: Aug. 27, 2026

(54) FREE-FLOATING REINFORCEMENT TAG FOR SURGICAL SUTURE MATERIAL

(71) Applicant: Winter Innovations, Inc., Knoxville, TN (US)

(72) Inventors: Lia Lynn Winter, Pittsburgh, PA (US); Preston Dishner, Piney Flats, TN (US); John Oberman, Knoxville, TN (US)

(73) Assignee: Winter Innovations, Inc., Knoxville, TN (US)

(21) Appl. No.: 19/552,267

(22) Filed: Feb. 27, 2026

Related U.S. Application Data
(60) Provisional application No. 63/764,279, filed on Feb. 27, 2025.
(60) Provisional application No. 63/764,978, filed on Feb. 28, 2025.

(51) Int. Cl. A61B 17/06; A61B 17/04

(52) U.S. Cl. CPC A61B 17/06166; A61B 17/06004; A61B 2017/0495; A61B 2017/06057

(57) ABSTRACT

A free-floating reinforcement tag for use with sewing materials, such as surgical suture material. The reinforcement tag includes a contact surface and is configured such that sewing material extends through the tag, allowing the tag to be free-floating and adjustably positionable along the length of the sewing material. The tag is positioned at a user-selected location and placed flat against a sewing surface, then secured by passing the sewing material through both the tag and the sewing surface. The tag distributes forces applied by the sewing material across the sewing surface to prevent tissue damage and shredding. The tag may be attached via pre-formed or needle-created apertures, or in a luggage tag configuration. The system is particularly useful for ligament and tendon repair and reconstruction surgeries, and may be used with multi-part needle assemblies and suspensory loop devices.

### CROSS-REFERENCE TO RELATED APPLICATIONS

This application claims priority to and the benefit of U.S. Provisional Patent Application No. 63/764,279, filed Feb. 27, 2025, titled “REINFORCEMENT TAG,” and U.S. Provisional Patent Application No. 63/764,978, filed Feb. 28, 2025, titled “FREE-FLOATING TAG FOR STITCH REINFORCEMENT,” both of which are incorporated herein by reference in their entirety.

### FIELD

The present invention relates to sewing devices and techniques, particularly to surgical suturing systems and methods. More specifically, the invention relates to a free-floating reinforcement tag that integrates with surgical suture material and can be adjustably positioned along the length of the suture material to reinforce biological tissue during surgical procedures.

### BACKGROUND

### Orthopedic Ligament and Tendon Surgery

Ligaments and tendons are soft tissue structures that commonly tear when too much force is applied to a joint. These injuries occur in many areas of the body, with the most common including tears of the anterior cruciate ligament (ACL), biceps tendon, and Achilles tendon. More than 1 million ligament and tendon surgeries are performed in the United States each year, with approximately 1.6 million annual procedures completed globally.

Surgery for a torn ligament or tendon relies on stitching the tissue with a needle and specialized high-strength sutures, but the process is technically complex. Depending on the nature and severity of the injury, a surgeon may either repair or reconstruct the tissue. For a repair, the native tissue is stitched back together. For a reconstruction, a replacement ligament or tendon (called a graft) is isolated from another part of the patient's body (autograft) or from a donor (allograft). The stitches serve to reinforce the tissue and allow proper tension to be placed for fixation.

### Efficiency and Biomechanical Security

Effective stitching requires two key elements: efficiency (the ability to stitch quickly, which is achieved with fewer needle passes) and biomechanical security (high strength and resistance to elongation). Efficiency is important because it results in reduced surgical time, which can afford cost savings from less anesthesia and boost revenue from an improved rate of operating room turnover with time for additional procedures. In addition to saving time, fewer needle passes result in less damage to the tissue. Security is important because secure repairs resist failure and can reduce the need for revision surgeries.

If stitching is not done properly, there is a high likelihood the procedure will fail, resulting in pain and instability that requires a revision surgery. Of the thousands of ligament and tendon surgeries performed each year, many require a revision due to problems with the graft or stitching. This represents a significant cost to the healthcare system.

### Stitch Patterns

A popular stitch pattern, called the whip stitch, is made with a loop suture needle. The whip stitch is known for its efficiency because one pass of the needle stitches both sides of the tissue simultaneously. However, the whip stitch has been shown to lack biomechanical security because it concentrates the force down the central axis of the tissue and commonly fails due to damage and shredding of the tissue.

There is a need for solutions to the security problems posed by the whip stitch. One approach is to stitch in locking-style patterns to spread the force across the tissue. These stitching patterns can improve the security of a graft and lead to a failure mode of suture breakage, which is a more favorable and reparable outcome for failure. The Krackow method is an example of a locking-style stitch, which has been proven to obtain strong biomechanical security in repaired or reconstructed tissue, but this method requires twice as many needle passes as a whip stitch, thereby decreasing the efficiency of the stitching process.

Multi-part needle systems have been developed that allow for both a whip stitch and locking-style patterns to be utilized on tissue for repair or reconstruction. Such systems allow for a locking-style stitch pattern to be used on the tissue while requiring the same number of needle passes as a whip stitch, thereby allowing for both efficiency and security.

### Fixation Techniques

After stitching, the next step in a ligament or tendon repair/reconstruction surgery is fixation, in which the tissue is secured in place, either to another ligament/tendon or to a bone.

When a stitched ligament/tendon is fixed to another ligament/tendon, it is commonly referred to as “mid-substance fixation.” In this surgical approach, the sutures used in stitching play a critical role in securing the repair, as they receive a significant amount of the force applied to the tissue under load. An example of mid-substance fixation is the repair of the Achilles tendon. In this process, the tendon rupture is isolated, and the torn ends of the tendon are sutured together to complete the repair.

When a stitched ligament/tendon is fixed to a bone, additional devices such as screws or suspensory loop buttons are used in conjunction. In this surgical approach, integration of the stitched graft with the chosen fixation device is critical to the security of a successful tissue reconstruction. For screw fixation, the stitches placed on a graft provide bite for the screw and serve to hold the tissue securely against the bone. For suspensory button fixation, the sutures are either tied around the button or are directly attached to an intermediary suspensory loop device.

An example of suspensory loop fixation is ACL reconstruction. In a typical ACL reconstruction surgery, a ligament or tendon graft is taken from another part of the body, usually the quadriceps or hamstrings. The surgeon then uses a needle and suture to attach the end of the graft to the suspensory loop device. This graft is then implanted into the knee, which involves passing the suspensory loop through a bone tunnel using a cortical button to hold the graft in place.

### Reinforcement Approaches

Stitching is crucial to the success of fixation in ligament/tendon repair and reconstruction. Commonly, the whip stitch is used in the stitching process due to its efficiency, but its lack of security poses concerns for failure. While locking stitches provide reinforcement and bolster security, the additional needle holes and stitching time also pose problems.

Another approach to reinforce the whip stitch is through the use of additional material. By stitching through extra material (referred to as a “tag”) in addition to the tissue, the material can effectively act as a “rip-stop” to disperse the force placed on the sutures and prevent tissue damage, shredding, and pull-through.

Various products exist that utilize additional material stitched through to reinforce the construct. Some systems use thicker suture material that forms the tag and is fixedly attached to the suture. A needle is attached to both tails of the suture, which is then used to whip stitch through both the tag and the tissue simultaneously.

Another approach to reinforcement is to utilize biologic materials (e.g., dermal graft, allograft tissue, etc.) or synthetic materials that mimic biologic structures and/or degrade over time. Various biomimetic reinforcement materials are available that are stitched through to reinforce repair/reconstruction of a variety of tendon or ligament injuries. In these cases, the suture is completely independent of the reinforcement material through which it is passed.

Existing products utilize a reinforcement tag that is either fixedly attached to the suture strands that are passed through it or completely independent of the suture. However, there is unique utility in a reinforcement tag that is free-floating on the suture and adjustably positionable along the length of the suture. Such a tag can be selectively positioned at any desired location along the suture length, including at a center point between needle ends or at other locations as needed for optimal reinforcement. This adjustability provides significant advantages over fixed-position tags or completely independent reinforcement materials.

Therefore, what is needed is a reinforcement tag that can be selectively adjusted and repositioned along the length of the suture material during the stitching process, providing both the biomechanical security of reinforcement without being fixedly attached to a single location on the suture or completely independent from it.

### Notes on Construction

The use of the terms “a”, “an”, “the” and similar terms in the context of describing the invention are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. The terms “substantially”, “generally” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic.

Terms concerning attachments, coupling and the like, such as “connected” and “interconnected”, refer to a relationship wherein structures are secured or attached to one another either directly or indirectly through intervening structures, as well as both moveable and rigid attachments or relationships, unless specified herein or clearly indicated by context. The term “operatively connected” is such an attachment, coupling or connection that allows the pertinent structures to operate as intended by virtue of that relationship.

The use of any and all examples or exemplary language (e.g., “such as” and “preferably”) herein is intended merely to better illuminate the invention and the preferred embodiment thereof, and not to place a limitation on the scope of the invention. Nothing in the specification should be construed as indicating any element as essential to the practice of the invention unless so stated with specificity.

### BRIEF DESCRIPTION OF THE DRAWINGS

Further advantages of the invention are apparent by reference to the detailed description when considered in conjunction with the figures, which are not to scale so as to more clearly show the details, wherein like reference numerals represent like elements throughout the several views, and wherein:

FIG. 1 illustrates various reinforcement tag configurations according to embodiments of the invention.

FIG. 2 shows integration of a needle with a reinforcement tag, wherein the needle creates an aperture in the reinforcement tag.

FIG. 3 shows integrations of a needle with a reinforcement tag having a pre-formed aperture.

FIG. 4 illustrates a reinforcement tag attached to a loop cord via a luggage tag configuration.

FIG. 5 illustrates a reinforcement tag having a single aperture attached to a loop cord and suspensory button via a luggage tag configuration.

FIG. 6 illustrates a reinforcement tag having a pair of apertures attached to a loop cord and suspensory button via a luggage tag configuration.

FIGS. 7A and 7B depict a first end of the reinforcement tag of FIG. 5 being attached to a graft with a length of sewing material.

FIGS. 8A and 8B depict a second end of the reinforcement tag of FIG. 5 being attached to a graft with a length of sewing material.

FIG. 9 depicts a plurality of reinforcement tags, each positioned at different user-selected locations along the length of a sewing material, and each reinforcement tag being free-floating and adjustably positionable, according to an embodiment of the present invention.

### DETAILED DESCRIPTION

Referring now to the drawings in which like reference characters designate like or corresponding characters throughout the several views, there is shown a free-floating reinforcement tag system for use in surgical and general sewing applications, particularly for reinforcing stitched tissue and other sewing materials to prevent damage and increase structural integrity.

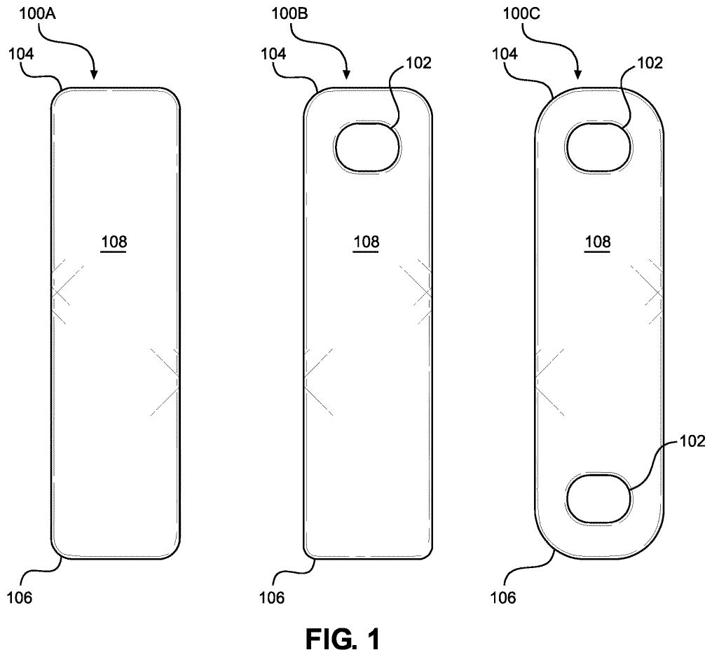

In particular, with reference to FIG. 1., reinforcement tags 100 according to various embodiments of the present invention are shown, including a first tag 100A that provides no apertures, a second tag 100B that provides a single aperture 102 located at a first end 104 of the tag, and a third tag 100C that provides apertures 102 located at the first end and at a second end 106 of the tag. In each case, the tag 100 provides a contact surface 108. The reinforcement tags 100 may be formed from various materials, including but not limited to braided suture material, suture tape, biological tissue, synthetic augmentation material, bioresorbable polymer, and combinations thereof.

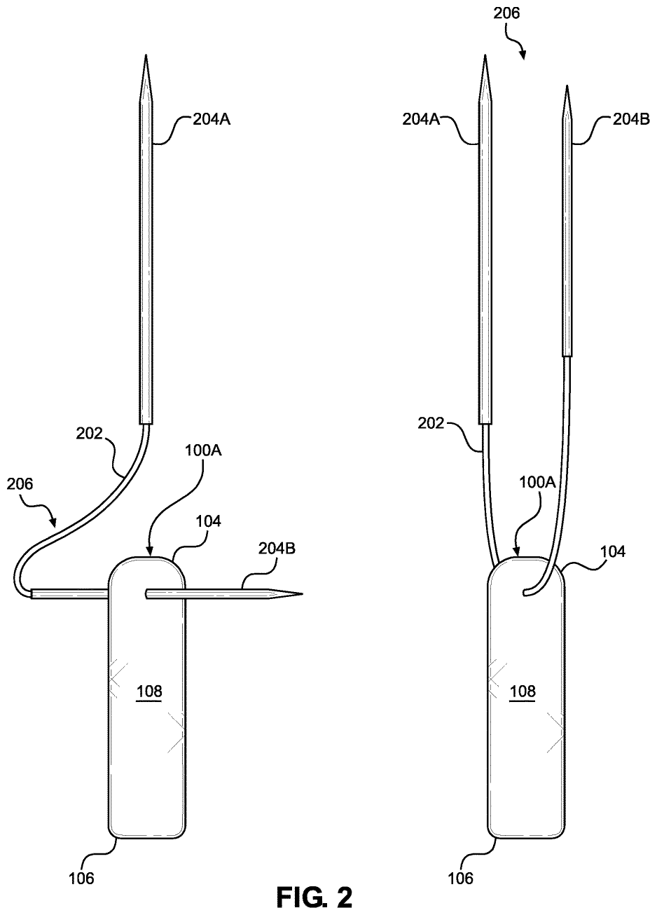

With continued reference to FIG. 1 and with further reference to FIG. 2, the first tag 100A is shown as part of a sewing material combination 200 that also includes a sewing material 202 having a length (e.g., a 24″ piece of suture material) that extends through the reinforcement tag 100 such that the reinforcement tag is free-floating and adjustably positionable along the length of the sewing material. In preferred embodiments, the sewing material 202 includes at least one needle (or needle portion 204A) attached to an end thereof. However, in the illustrated embodiment, a multi-part needle assembly 206 is in use. The assembly 206 includes the first needle portion 204A along with a second needle portion 204B that can be selectively coupled together to form a combined needle configuration and separable to form a separated needle configuration while remaining connected via the sewing material. Suitable needle assemblies and methods of use are described in U.S. Pat. No. 11,213,290, the content of which is hereby incorporated by reference in its entirety.

In the illustrated embodiment, the reinforcement tag 100A is provided with no apertures. Instead, the entire contact surface 108 is solid (i.e., aperture-less). Therefore, when the sewing material combination 200 is in use, in order to pass the sewing material 202 through the reinforcement tag 100A, a needle (or needle portion 204A, 204B) is used to pierce the tag to form an aperture through which the sewing material extends. The tag 100A is then configured to be positioned at a user-selected location along the length of the sewing material 202 and the contact surface 108 is configured to be positioned flat against a sewing surface (e.g., a graft, a patient, etc.) and secured to the sewing surface by passing the sewing material through both the reinforcement tag and the sewing surface to distribute forces applied by the sewing material across the sewing surface. In preferred embodiments, the contact surface 108 is planar so as to facilitate flat contact with the sewing surface.

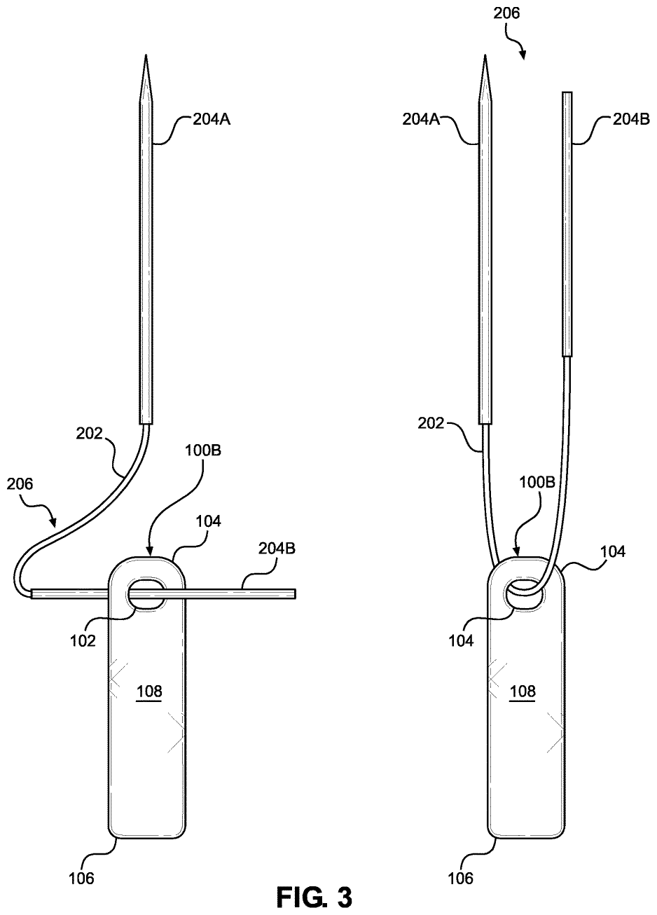

Now, with reference to FIG. 3, an alternative embodiment is shown in which the reinforcement tag 100B includes a pre-formed aperture 102 located at the first end 104 of the tag. In this embodiment, rather than piercing the tag with a needle to create an aperture, the needle (or needle portion 204A, 204B) is passed through the pre-existing aperture 102 to thread the sewing material 202 through the tag. This configuration allows for repeated adjustment and repositioning of the tag along the length of the sewing material without creating additional apertures in the tag material.

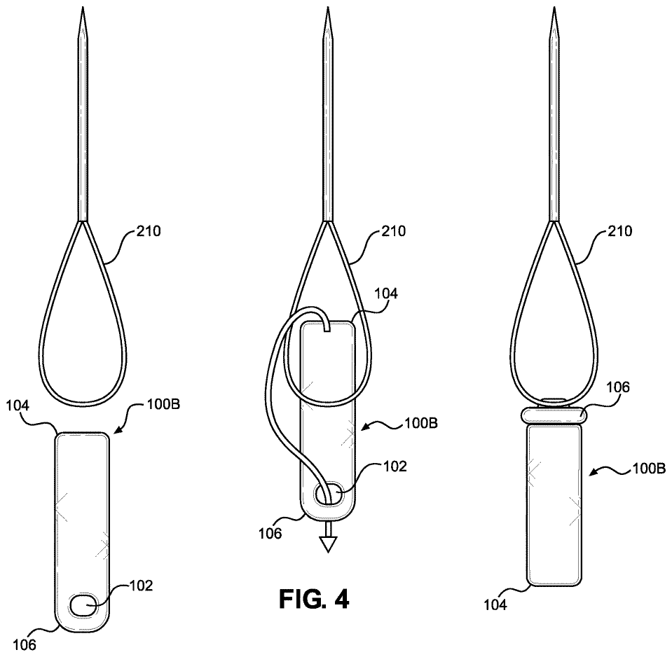

With reference to FIGS. 4-6, alternative attachment methods are illustrated in which various reinforcement tags 100 are attached using a “luggage tag” configuration. In these embodiments, the tag 100 is wrapped around a loop cord 210 (e.g., an example of a suspensory loop device) and one end 104, 106 of the tag is inserted through an aperture 102 at the opposite end of the tag to form a closed loop. This configuration is particularly advantageous when the tag is integrated with a loop cord 210 (FIG. 4) and/or with a fixation device 212 (e.g., a cortical button).

As shown in FIG. 4, the reinforcement tag 100B having a single aperture 102 at the second end 106 may be wrapped around a loop cord 210 such that the first end 104 of the tag is inserted through the aperture 102 to form a closed loop. This luggage tag configuration allows the reinforcement tag 100B to remain securely positioned on the loop cord 210 while still permitting adjustment of the tag's position along the length of the loop cord.

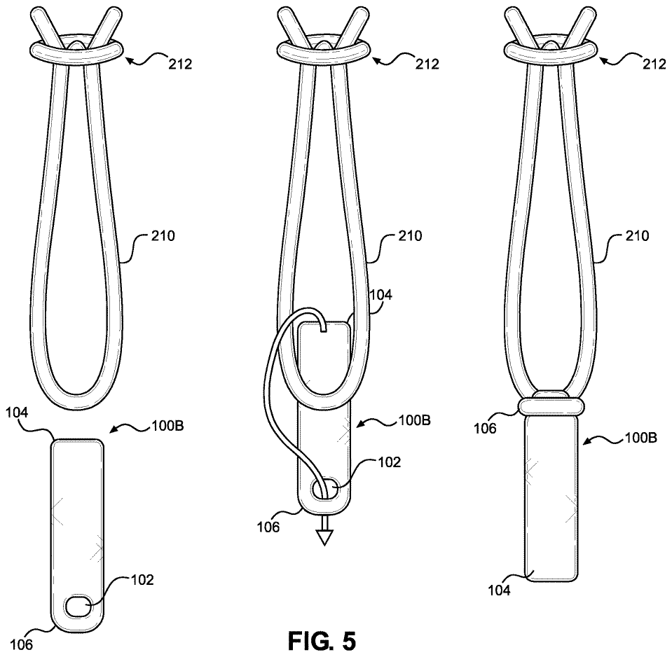

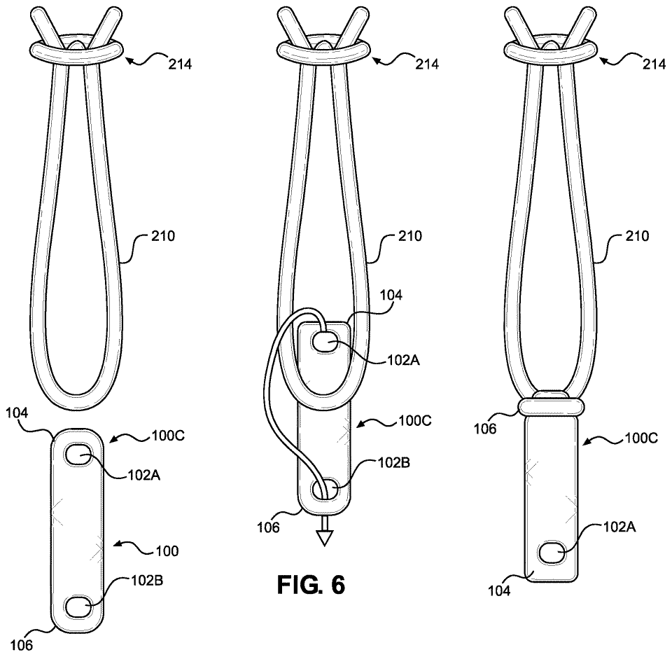

Similarly, with reference to FIG. 5, the reinforcement tag 100B is shown integrated with a loop cord 210 and a cortical button 214 via the luggage tag configuration. In this embodiment, the reinforcement tag 100B is wrapped around the loop cord 210 and secured by inserting the first end 104 through the aperture 102 at the second end 106 to form a closed loop. Similarly, FIG. 6 illustrates an alternative embodiment in which the reinforcement tag 100C includes apertures 102 at both the first end 104 and the second end 106. This dual-aperture configuration provides additional flexibility in how the reinforcement tag 100C is attached to the loop cord 210 and may provide enhanced structural integrity depending on the specific surgical application. As in the prior embodiment, the reinforcement tag 100C is wrapped around the loop cord 210 and secured by inserting the first end 104 through the aperture 102 at the second end 106 to form a closed loop. In each case, the cortical button 214 is shown connected to the loop cord 210, providing an example of how the reinforcement tag may be integrated into a suspensory fixation system for surgical applications such as ACL reconstruction.

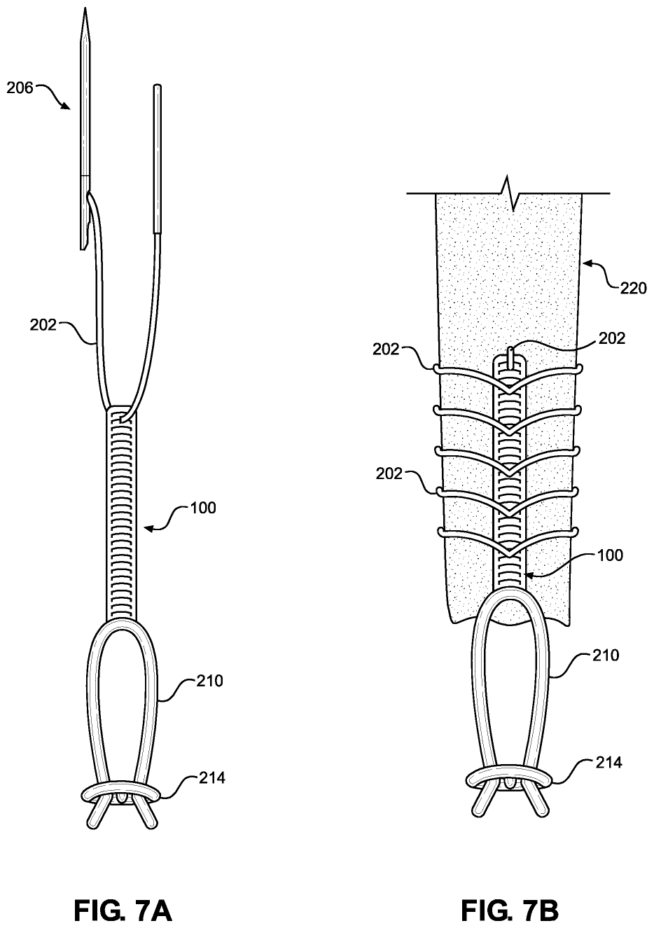

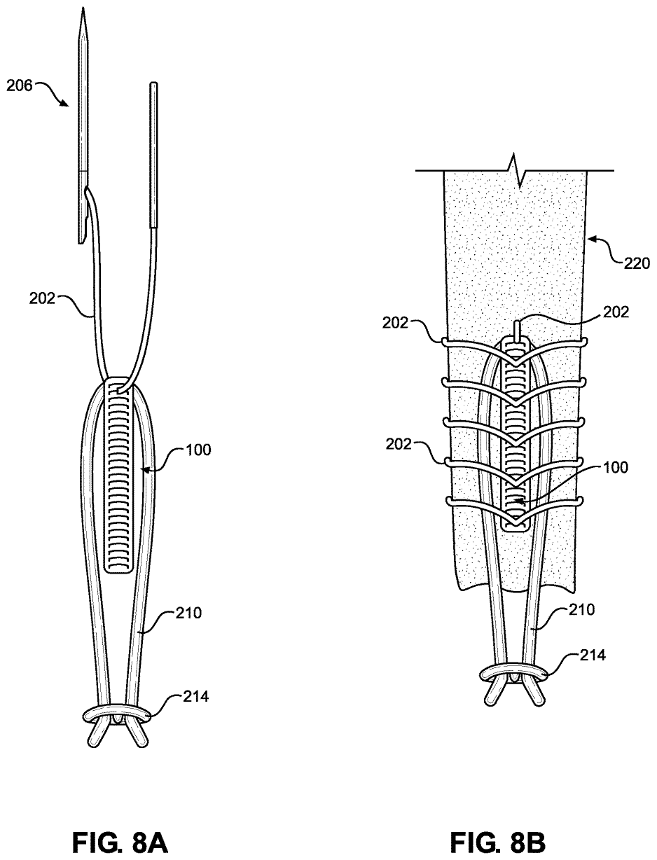

With reference to FIGS. 7A-7B and FIGS. 8A-8B, respectively, two different attachment configurations of the reinforcement tag system during a surgical procedure are illustrated. The key difference between these configurations is the location of attachment of the sewing material 202 and loop cord 210 relative to the ends 104, 106 of the reinforcement tag 100. As shown in FIGS. 7A and 7B, a first attachment configuration is illustrated where the sewing material 202 initially engages (i.e., the first stitch is formed) one end of the tag 100 and the loop cord 210 is attached to an opposite end of the tag. The reinforcement tag 100 is positioned flat against the sewing surface 220 (such as a graft or tissue), and the sewing material 202 is passed through both the tag and the sewing surface using the needle assembly 206 to fixedly attach the tag to the sewing surface.

As shown in FIGS. 8A and 8B, a second attachment configuration is illustrated where the sewing material 202 initially engages (i.e., the first stitch is formed) at one end of the tag 100 and the loop cord 210 is attached to the same end of the tag. Similar to the first configuration, the reinforcement tag 100 is then positioned flat against the sewing surface 220, and the sewing material 202 is passed through both the tag and the sewing surface to complete the attachment.

In the two configurations discussed above, the attachment configuration and the placement of the tag 100 on the sewing material 202 dictate the direction of stitching. In both configurations, the resulting construction provides reinforcement across the sewing surface, with the tag 100 acting as a “rip-stop” to distribute forces applied by the sewing material 202 and prevent the sewing material from shredding or damaging the sewing surface. In each case, the sewing material 202 may be passed through the tag 100 and sewing surface 220 using various stitching methods known in the art.

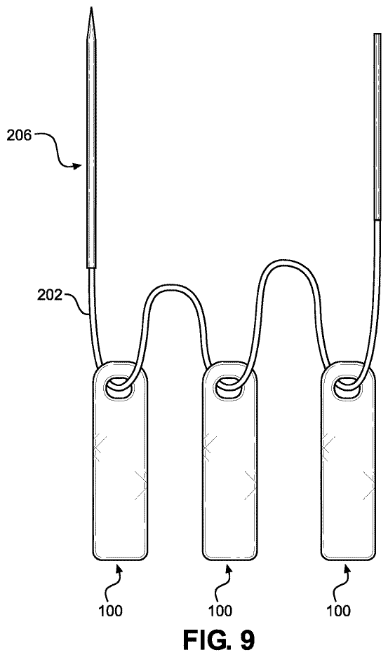

Finally, with reference to FIG. 9, an embodiment is illustrated in which a plurality of reinforcement tags 100 are positioned at different user-selected locations along the length of the sewing material 202. Each reinforcement tag is free-floating and adjustably positionable, allowing a user to customize the reinforcement pattern based on the specific requirements of the surgical procedure. This multi-tag configuration is particularly advantageous for reinforcing longer sections of tissue or for providing reinforcement at multiple critical points along a graft.

In certain embodiments, the reinforcement tag may comprise a tubular sleeve configuration (not shown) in which the sewing material extends through a hollow interior of the sleeve. In this configuration, rather than passing the sewing material through discrete apertures, the sewing material is threaded through the length of the tubular sleeve, and the sleeve remains free-floating and adjustably positionable along the length of the sewing material.

The sewing surface 220 referenced throughout this disclosure may comprise any material that can be sutured, including but not limited to biological tissues (such as ligaments, tendons, muscles, skin, and other soft tissues), grafts (both autografts and allografts), synthetic materials, and combinations thereof. The reinforcement tag system of the present invention is particularly advantageous for surgical applications involving ligament and tendon repair or reconstruction, but may also be utilized in other surgical and non-surgical sewing applications where reinforcement is desired.

The adjustable, free-floating nature of the reinforcement tag provides significant advantages over prior art systems in which reinforcement tags are either fixedly attached to a predetermined location on the sewing material or are completely independent of the sewing material. The ability to selectively position and reposition the tag along the length of the sewing material allows a surgeon to optimize the placement of reinforcement based on the specific anatomy and surgical requirements of each individual case, thereby improving surgical outcomes and reducing the risk of tissue damage and failure.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations as would be appreciated by those having ordinary skill in the art to which the invention relates

What is claimed is:

1. A sewing material combination comprising:
sewing material having a length; and
a reinforcement tag having a contact surface, wherein the sewing material extends through the reinforcement tag such that the reinforcement tag is free-floating and adjustably positionable along the length of the sewing material;
wherein the reinforcement tag is configured to be positioned at a user-selected location along the length of the sewing material and the contact surface is configured to be positioned flat against a sewing surface and secured to the sewing surface by passing the sewing material through both the reinforcement tag and the sewing surface.

2. The sewing material combination of claim 1, wherein the contact surface is planar so as to make flat contact with the sewing surface.

3. The sewing material combination of claim 1, wherein the sewing material comprises at least one needle attached to an end thereof.

4. The sewing material combination of claim 3, wherein the at least one needle comprises a multi-part needle assembly having a first needle portion and a second needle portion that are selectively couplable to form a combined needle configuration and separable to form a separated needle configuration while remaining connected via the sewing material.

5. The sewing material combination of claim 1, wherein the reinforcement tag comprises a material selected from the group consisting of: braided suture material, suture tape, biological tissue, synthetic augmentation material, bioresorbable polymer, and combinations thereof.

6. The sewing material combination of claim 1, wherein the reinforcement tag comprises a tubular sleeve through which the sewing material extends.

7. The sewing material combination of claim 1, further comprising a suspensory loop device.

8. The sewing material combination of claim 1, further comprising a plurality of reinforcement tags, each positioned at different user-selected locations along the length of the sewing material, and each reinforcement tag being free-floating and adjustably positionable.

9. A method of reinforcing a sewing surface, the method comprising:
providing sewing material having a length and at least one needle attached to an end thereof;
providing a reinforcement tag having a contact surface;
passing the sewing material through the reinforcement tag such that the reinforcement tag is free-floating and adjustably positionable along the length of the sewing material;
adjusting a position of the reinforcement tag to a user-selected location along the length of the sewing material;
positioning the contact surface of the reinforcement tag flat against a sewing surface; and
passing the sewing material through both the reinforcement tag and the sewing surface to fixedly attach the reinforcement tag to the sewing surface, wherein the contact surface distributes forces applied by the sewing material across the sewing surface.

10. The method of claim 9, wherein the contact surface is planar so as to make flat contact with the sewing surface.

11. The method of claim 9, wherein passing the sewing material through the reinforcement tag comprises passing the at least one needle through a preformed aperture in the reinforcement tag.

12. The method of claim 9, wherein passing the sewing material through the reinforcement tag comprises piercing the reinforcement tag with the at least one needle to form an aperture through which the sewing material extends.

13. The method of claim 9, wherein passing the sewing material through the reinforcement tag comprises wrapping the reinforcement tag around the sewing material and inserting a first end of the reinforcement tag through an aperture at a second end of the reinforcement tag to form a closed loop around the sewing material.

14. The method of claim 9, wherein the at least one needle comprises a multi-part needle assembly having a first needle portion and a second needle portion that are selectively couplable to form a combined needle configuration, and wherein passing the sewing material through both the reinforcement tag and the sewing surface is performed with the multi-part needle assembly in the combined needle configuration.

15. The method of claim 9, wherein the reinforcement tag comprises a material selected from the group consisting of: braided suture material, suture tape, biological tissue, synthetic augmentation material, bioresorbable polymer, and combinations thereof.

16. The method of claim 9, wherein the sewing material comprises a looped portion retained by a suspensory loop device, and wherein passing the sewing material through the reinforcement tag comprises wrapping the reinforcement tag around the looped portion and inserting a first end of the reinforcement tag through an aperture at a second end to form a closed loop around the looped portion.

17. The method of claim 9, wherein the tag functions as a rip-stop to distribute forces and prevent the sewing material from shredding the sewing surface.

18. A kit for surgical reinforcement comprising:
sewing material having a length and at least one needle attached to an end thereof; and
at least one reinforcement tag having a contact surface and configured to receive the sewing material therethrough such that the reinforcement tag is free-floating and adjustably positionable to a user-selected location along the length of the sewing material;
wherein the contact surface is configured to be positioned flat against a sewing surface and to distribute forces applied by the sewing material when the reinforcement tag is secured to the sewing surface.

19. The kit of claim 18, wherein the contact surface is planar so as to make flat contact with the sewing surface.

20. The kit of claim 18, wherein the at least one needle comprises a multi-part needle assembly having a first needle portion and a second needle portion that are selectively couplable and separable while remaining connected via the sewing material.
